(19) United States
(12) Patent Application Publication
(10) Pub. No.: US 2025/0276003 A1
(43) Pub. Date: Sep. 4, 2025

(54) PARMELIA TINCTORUM POLYSACCHARIDE XEP-70, METHOD OF PREPARATION, AND USE THEREOF

(71) Applicant: NORTHWEST INSTITUTE OF PLATEAU BIOLOGY, CHINESE ACADEMY OF SCIENCES, Xining (CN)

(72) Inventors: Hongxia YANG, Xining (CN); Hongtao BI, Xining (CN); Zheng ZHOU, Xining (CN); Guoqiang LI, Xining (CN); Liang GAO, Xining (CN); Yubi ZHOU, Xining (CN); Yuancan XIAO, Xining (CN)

(21) Appl. No.: 18/904,132

(22) Filed: Oct. 2, 2024

Related U.S. Application Data
(63) Continuation of application No. PCT/CN2024/082837, filed on Mar. 21, 2024.

(30) Foreign Application Priority Data
Mar. 1, 2024 (CN) 202410238888.6

(51) Int. Cl. A61K 31/715; A61K 36/09; A61P 39/06; C08B 37/00

(52) U.S. Cl. CPC A61K 31/715; A61K 36/09; A61P 39/06; C08B 37/0003

(57) ABSTRACT

The present disclosure belongs to the field of natural high polymers and biopharmaceutical technology, and specifically relates to a Parmelia tinctorum polysaccharide XEP-70, a method of preparation, and use thereof. The sugar residues and glycosidic bonds of the polysaccharide XEP-70 provided in the present disclosure include: (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf., with an weight-average molecular weight of 430.98 kDa. The polysaccharide may enhance cellular antioxidant capacity by activating the Nrf2-Keap1-ARE signaling pathway, effectively protecting LX-2 cells from oxidative damage induced by hydrogen peroxide.

### CROSS-REFERENCE TO RELATED APPLICATIONS

This application is a continuation of International Patent Application No. PCT/CN2024/082837, filed on Mar. 21, 2024, which claims the priority of Chinese Patent Application No. CN202410238888.6 filed with the China National Intellectual Property Administration on Mar. 1, 2024, both of which are incorporated by reference herein in its entirety as part of the present application. The entire disclosures of the above applications are incorporated herein by reference.

### FIELD

The present technology relates to the field of natural polymer and biopharmaceutical technology, and specially relates to a Parmelia tinctorum polysaccharide XEP-70, a method of preparation, and use thereof.

### INTRODUCTION

This section provides background information related to the present disclosure which is not necessarily prior art.

In biological systems, an increase in reactive oxygen species (ROS) levels and a decrease in antioxidant mechanisms can lead to cellular oxidative stress. Under normal circumstances, ROS is generated during mitochondrial oxygen metabolism and participates in cell signaling. However, excessive ROS production and impaired antioxidant capacity can induce structural damage to cells and disrupt tissues. This is believed to be associated with the occurrence of many chronic diseases such as inflammation, diabetes, cancer, and aging.

Hydrogen peroxide is one of the main causes of cellular oxidative stress and is commonly used to assess antioxidant capacity and the ability of cells to clear ROS. Studies (D. K. Ingawale, S. K. Mandlik, S. R. Naik, Models of hepatotoxicity and the underlying cellular, biochemical and immunological mechanism(s): a critical discussion, Environ Toxicol Pharmacol 37(1) (2014) 118-33; M. Parola, G. Robino, Oxidative stress-related molecules and liver fibrosis, J Hepatol 35(2) (2001) 297-306.) have shown that liver cell damage is closely related to increased ROS, and oxidative stress is considered one of the pathological mechanisms leading to the occurrence and development of various liver diseases. Therefore, antioxidants are considered a good strategy for treating liver diseases.

Although artificially synthesized antioxidants can inhibit the chain reaction of oxidative free radicals, they inevitably produce side effects and may lead to liver damage and carcinogenesis. Therefore, the development of natural antioxidants that can protect the human body from free radical damage and delay the progression of liver diseases is crucial.

### SUMMARY

It is an objective of the present disclosure to provide a Parmelia tinctorum polysaccharide XEP-70, a method of preparation, and use thereof. Parmelia tinctorum polysaccharide XEP-70 is a natural polymer that can enhance cellular antioxidant capacity, effectively protecting against oxidative damage, particularly protecting liver cells from oxidative damage induced by hydrogen peroxide.

To achieve the above objective, the present disclosure provides a Parmelia tinctorum polysaccharide XEP-70 having sugar residues and glycosidic bonds including: (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf.; where
the Parmelia tinctorum polysaccharide XEP-70 has a weight-average molecular weight of 430.98 kDa.

In some embodiments, the Parmelia tinctorum polysaccharide XEP-70 has a total sugar content of 99.05 wt. % and a glucuronic acid content of 13.09 wt. %.

In some embodiments, monosaccharides of the Parmelia tinctorum polysaccharide XEP-70 consist of mannose, galacturonic acid, glucose, galactose, and xylose.

In some embodiments, the mannose, the galacturonic acid, the glucose, the galactose, and the xylose have a molar ratio of 39.28:16.09:17.56:23.20:3.87.

The present disclosure further provides a method for preparing the Parmelia tinctorum polysaccharide XEP-70 as described above, including the following steps:
subjecting Parmelia tinctorum to thermal extraction, followed by solid-liquid separation and concentration to obtain a crude polysaccharide solution;
subjecting the crude polysaccharide solution to ethanol precipitation, collecting a resulting precipitate, and conducting lyophilization to obtain a crude Parmelia tinctorum polysaccharide;
performing separation and purification of the crude Parmelia tinctorum polysaccharide using a diethylaminoethyl (DEAE) anion exchange column, where the separation and purification include re-dissolving the crude Parmelia tinctorum polysaccharide in water to make an aqueous solution, loading the aqueous solution on the DEAE anion exchange column, eluting with water and 0.5 mol/L NaCl solution in sequence, collecting an eluate of 0.5 mol/L NaCl solution, subjecting the eluate to dialysis, and lyophilizing the dialyzed solution to obtain a Parmelia tinctorum polysaccharide XEP-1; and
re-dissolving the Parmelia tinctorum polysaccharide XEP-1, adding anhydrous ethanol to a final concentration of 70% v/v, allowing to stand at 0 to 4° C. for 8 to 48 h, collecting a pellet by centrifugation, subjecting the pellet to drying to obtain the Parmelia tinctorum polysaccharide XEP-70.

In some embodiments, the Parmelia tinctorum is subjected to thermal extraction with hot water three times each with a mass-volume ratio of 1 kg:10 L;
the thermal extraction with hot water is at a temperature of 90-100° C.;
the concentration is performed at 95° C.;
the ethanol precipitation is conducted using ethanol at a final concentration of 80% v/v;
the dialysis includes distilled water dialysis with a molecular weight cut-off of 3500 Da for 24 h and flowing water dialysis with a molecular weight cut-off of 1000-10000 Da for 24 h; and
the lyophilization is performed at a pressure of 10 to 30 MPa and a temperature of −60 to −80° C.

The present disclosure further provides use of the Parmelia tinctorum polysaccharide XEP-70 in the preparation of an antioxidant product.

In some embodiments, the antioxidant product includes a drug for preventing and/or treating oxidative damage.

In some embodiments, the Parmelia tinctorum polysaccharide XEP-70 in the drug has an effective concentration of 25 to 100 μg/mL.

In some embodiments, the oxidative damage includes damage induced by H2O2.

### Beneficial Effects

In the present disclosure, a polysaccharide XEP-70 is obtained from Parmelia tinctorum, and the sugar residue and glycosidic bonds of this polysaccharide include: (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf., with a weight-average molecular weight of 430.98 kDa. The polysaccharide XEP-70 is found to enhance cellular antioxidant capacity, effectively protecting against oxidative damage, particularly protecting liver cells from oxidative damage induced by hydrogen peroxide. In the present disclosure, the potential protective effect of XEP-70 on liver cell oxidative damage is evaluated using a hydrogen peroxide-induced oxidative damage model in LiemingXu-2 cells (LX-2). The results indicate that the polysaccharide XEP-70 from Parmelia tinctorum enhances cellular antioxidant capacity by activating the Nrf2-Keap1-ARE signaling pathway, effectively protecting LX-2 cells from oxidative damage induced by hydrogen peroxide. Compared to other polysaccharides from Parmelia tinctorum, XEP-70 exhibits the best antioxidant activity in free radical scavenging experiments, making it a potential natural antioxidant.

Further areas of applicability will become apparent from the description provided herein. The description and specific examples in this summary are intended for purposes of illustration only and are not intended to limit the scope of the present disclosure.

### DRAWINGS

In order to provide a clearer explanation of the technical solutions in the examples of the present application or the prior art, a brief description of the drawings required in the examples are provided.

FIG. 1 illustrates uniformity and molecular weight determination results of different Parmelia tinctorum polysaccharides;

FIG. 2 illustrates Fourier transform infrared (FTIR) spectroscopy analysis results of different Parmelia tinctorum polysaccharides;

FIGS. 3A-3B illustrate scanning electron microscopy (SEM) analysis results of Parmelia tinctorum polysaccharide XEP-70; where FIG. 3A is magnified at 400 times, and FIG. 3B is magnified at 4000 times;

FIGS. 4A-4B illustrates atomic force microscopy (AFM) analysis results of Parmelia tinctorum polysaccharide XEP-70; where FIG. 4A is a two-dimensional image, and FIG. 4B is a three-dimensional image;

FIG. 5 illustrates the nuclear magnetic resonance analysis results (1H NMR) of Parmelia tinctorum polysaccharide XEP-70;

FIG. 6 illustrates nuclear magnetic resonance analysis results (13C NMR) of Parmelia tinctorum polysaccharide XEP-70;

FIGS. 7A-7E illustrate test results of the ability of different Parmelia tinctorum polysaccharides to scavenge free radicals; where all data are represented as mean±SD (n=4), FIG. 7A-FIG. 7D correspond to 1,1-diphenyl-2-picryl-hydrazyl (DPPH) radicals, 2,2′-Azinobis-(3-ethylbenzthiazoline-6-sulphonate) (ABTS) radicals, O2- radicals, and OH— radicals respectively; FIG. 7E represents reducing power;

FIG. 8 illustrates impact of different concentrations of Parmelia tinctorum polysaccharide XEP-70 on LX-2 cell viability; where all data are represented as mean±SD (n=6), compared to H2O2, *p<0.05, **p<0.01;

FIG. 9 illustrates effect of different concentrations of H₂O₂ on LX-2 cell viability; where all data are represented as mean±SD (n=6), compared to H2O2, *p<0.05, **p<0.01, ***p<0.001;

FIG. 10 illustrates the antioxidant activity detection results (cell viability) of different concentrations of Parmelia tinctorum polysaccharide XEP-70 in the H₂O₂ oxidative damage model; where all data are represented as mean±SD (n=4), compared to H₂O₂, *p<0.05, **p<0.01, ***p<0.001;

FIGS. 11A-11G illustrate results of oxidative stress level and antioxidant enzyme activity detection of different concentrations of Parmelia tinctorum polysaccharide XEP-70 in the H₂O₂ oxidative damage model; FIGS. 11A-11G represent malondialdehyde (MDA) content, lactate dehydrogenase (LDH) activity, superoxide dismutase (SOD) activity, total antioxidant capacity (T-AOC) activity, glutathione (GSH) activity, SOD activity, and catalase (CAT) activity, all data are represented as mean±SD (n=4), compared to H₂O₂, *p<0.05, **p<0.01, ***p<0.001;

FIG. 12 illustrates results of protein immunoblotting detection;

FIGS. 13A-13F illustrate impact of Parmelia tinctorum polysaccharide XEP-70 on the expression of key proteins in the Nrf2-Keap1-ARE signaling pathway in the H₂O₂ oxidative damage model; compared to H2O2, *p<0.05, **p<0.01, ***p<0.001.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

As referred to herein, all compositional percentages are by weight of the total composition, unless otherwise specified. Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

The present disclosure provides a Parmelia tinctorum polysaccharide XEP-70 having sugar residues and glycosidic bonds including: (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf.; where
the Parmelia tinctorum polysaccharide XEP-70 has a molecular weight of 430.98 kDa.

In the present disclosure, the Parmelia tinctorum polysaccharide XEP-70 has a total sugar content of preferably 99.05 wt. %, and a glucuronic acid content of preferably 13.09 wt. %; monosaccharides of the Parmelia tinctorum polysaccharide XEP-70 preferably consist of mannose, galactose, glucose, galactose, and xylose; and the mannose, the galactose, the glucose, galactose, and the xylose have a molar ratio of preferably 39.28:16.09:17.56:23.20:3.87.

The present disclosure further provides a method for preparing the Parmelia tinctorum polysaccharide XEP-70 as described above, including the following steps:
subjecting Parmelia tinctorum to thermal extraction, followed by solid-liquid separation and concentration to obtain a crude polysaccharide solution;
subjecting the crude polysaccharide solution to ethanol precipitation, collecting a resulting precipitate, and conducting lyophilization to obtain a crude Parmelia tinctorum polysaccharide;
performing separation and purification of the crude Parmelia tinctorum polysaccharide using a diethylaminoethyl cellulose (DEAE) anion exchange column, where the separation and purification include re-dissolving the crude Parmelia tinctorum polysaccharide in water to make an aqueous solution, loading the aqueous solution on the DEAE anion exchange column, eluting with water and 0.5 mol/L NaCl solution in sequence, collecting an eluate of 0.5 mol/L NaCl solution, subjecting the eluate to dialysis, and lyophilizing the dialyzed solution to obtain a Parmelia tinctorum polysaccharide XEP-1; and
re-dissolving the Parmelia tinctorum polysaccharide XEP-1 with water, adding anhydrous ethanol to a final concentration of 70% v/v, allowing to stand at 0 to 4° C. for 8 to 48 h, collecting a pellet by centrifugation, subjecting the pellet to drying to obtain the Parmelia tinctorum polysaccharide XEP-70.

In the present disclosure, the Parmelia tinctorum is subjected to thermal extraction, followed by solid-liquid separation and concentration to obtain a crude polysaccharide solution. In the present disclosure, the number of thermal extractions is preferably 3 times, the mass-to-volume ratio of Parmelia tinctorum to water is preferably 1 kg:10 L for each hot extraction. The temperature of the thermal extraction is preferably 90-100° C., more preferably 95° C.; the temperature of the concentration is preferably 95° C.; and the concentration factor is preferably 9 times. The solid-liquid separation is conducted preferably by filtration. In the present disclosure, after the crude Parmelia tinctorum polysaccharide solution is obtained, the crude Parmelia tinctorum polysaccharide solution is subjected to ethanol precipitation, and the precipitate is collected and lyophilized to obtain the crude Parmelia tinctorum. In the present disclosure, the final concentration of alcohol precipitation is preferably 80% v/v. The collection of the precipitate is preferably performed by centrifugation; the centrifugation is preferably at a speed of 4500 rpm for preferably 15 min. The pressure for the lyophilization is preferably 10 to 30 MPa, more preferably 15 to 25 MPa, even more preferably 20 MPa; the temperature for the lyophilization is preferably −60 to −80° C., more preferably −70° C.

In the present disclosure, after the crude Parmelia tinctorum polysaccharide is obtained, the crude Parmelia tinctorum polysaccharide is separated and purified by DEAE anion exchange chromatography, and re-dissolved in water to prepare an aqueous solution. The aqueous solution is loaded onto a DEAE anion exchange column, eluted successively with water and 0.5 mol/L NaCl solution, and the eluted fraction of 0.5 mol/L NaCl solution is collected. In the present disclosure, the DEAE anion exchange column preferably includes a DEAE cellulose column. The crude Parmelia tinctorum polysaccharide and water are preferably mixed in a mass-to-volume ratio of 4 mg:1 mL to prepare the aqueous solution. The volume ratio of the aqueous solution, water for elution, and 0.5 mol/L NaCl solution for elution is preferably 1:40:40. The water used is preferably distilled water. By eluting with 0.5 mol/L NaCl solution, acidic polysaccharides can be obtained while removing strongly polar impurities.

In the present disclosure, after the eluate of a 0.5 mol/L NaCl salt solution is obtained, the eluate of the 0.5 mol/L NaCl solution is subjected to dialysis to obtain a Parmelia tinctorum polysaccharide XEP-1. In the present disclosure, the dialysis preferably includes distill water dialysis and flowing water dialysis. In the present disclosure, the components in NaCl solution are preferably sequentially subjected to distill water dialysis and flowing water dialysis; the molecular weight cut-off for distill water dialysis is preferably 3500 Da; the distill water dialysis is preferably performed for 24 h, more preferably changing to fresh distill water dialysis every 3 h; the molecular weight cut-off for the flowing water dialysis is preferably 1000 to 10000 Da, more preferably 2000 to 8000 Da, even more preferably 3000 to 6000 Da, and most preferably 3500 Da; the time for the flowing water dialysis is preferably 24 h. The preferred method of flowing water dialysis in the present disclosure is to use tap water at a certain flow rate; and the flow rate of tap water is preferably 300 mL/min. The pressure for the lyophilization in the present disclosure is preferably 10 to 30 MPa, more preferably 15 to 25 MPa, and even more preferably 20 MPa; the temperature for lyophilization is preferably −60 to −80° C., and more preferably −70° C.

In the process of preparing the polysaccharide XEP-70 according to the present disclosure, the crude Parmelia tinctorum polysaccharide is obtained with a yield of 8.62% through thermal extraction and alcohol precipitation. The crude Parmelia tinctorum polysaccharide is further purified into a 0.5 mol/L NaCl fraction XEP-1 with a yield of 2.48% using DEAE anion exchange column; purification of XEP-1 using ethanol precipitation method yields XEP-70 with a yield of 0.83%. In the present disclosure, after XEP-70 is obtained, the structural characteristics of XEP-70 is analyzed using nuclear magnetic resonance (NMR), SEM, and AFM, revealing sugar residues and glycosidic bonds including: (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf., with an weight-average molecular weight of 430.98 kDa. The potential protective effect of XEP-70 on oxidative damage to liver cells is evaluated using hydrogen peroxide-induced LiemingXu-2 cells (LX-2) oxidative damage model, demonstrating that the obtained polysaccharide XEP-70 may enhance cellular antioxidant capacity by activating the Nrf2-Keap1-ARE signaling pathway, effectively protecting LX-2 cells from hydrogen peroxide-induced oxidative damage. Compared to other polysaccharides, XEP-70 exhibits the best antioxidant activity in free radical scavenging experiments, making it a potential natural antioxidant.

Given the actions of the Parmelia tinctorum polysaccharide XEP-70 provided in the present disclosure, use of the polysaccharide XEP-70 in the preparation of an antioxidant product also falls within the scope of the present disclosure. In the present disclosure, the antioxidant product preferably includes a drug for preventing and/or treating oxidative damage; the oxidative damage preferably includes oxidative damage to liver cells; the oxidative damage preferably includes H₂O₂-induced oxidative damage; and the effective concentration of the polysaccharide XEP-70 in the drugs is preferably 25 to 100 μg/mL, and more preferably 100 g/mL. The oxidative damage model of hydrogen peroxide-induced LiemingXu-2 cells (LX-2) is established to evaluate the potential protective effect of the Parmelia tinctorum polysaccharide XEP-70 on oxidative damage to liver cells. The results show that XEP-70 may enhance cellular antioxidant capacity by activating the Nrf₂-Keap1-ARE signaling pathway, effectively protecting LX-2 cells from hydrogen peroxide-induced oxidative damage.

To further illustrate the present disclosure, the detailed description of the Parmelia tinctorum polysaccharide XEP-70, the method of preparation, and the use thereof provided in the present disclosure are described in conjunction with the drawings and examples. However, these should not be understood as limiting the scope of the present disclosure.

### Example 1

Preparation of Parmelia tinctorum Polysaccharides

### 1. Materials and Reagents

The Parmelia tinctorum was collected from the habitat of the Laji Mountain in Qinghai Province, China; DEAE cellulose was obtained from Yuanye BioTech Co., Ltd. (Shanghai, China); Bicinchoninic acid (BCA) and T-AOC kits were purchased from BioTeke Co., Ltd. (Shanghai, China); CAT, MDA, ROS, GSH, LDH, and SOD kits were purchased from Nanjing Jiancheng Bioengineering Institute (Nanjing, China); LX-2 cells (CL-0560) and Dulbecco's Modified Eagle Medium (DMEM) (PM1500210) were provided by Procell Corporation (Wuhan, China); FBS (10099-141C) was supplied by Gibco Corporation (New York, USA);

Except for the goat anti-rabbit antibody (HRP) (AS1107) purchased from Aspen Corporation (Wuhan, China), all antibodies used in the experiment were from Abcam Corporation (Cambridge, UK). All other chemicals were of analytical grade.

2. Extraction and Purification of Parmelia tinctorum Polysaccharides
(1) The Parmelia tinctorum was extracted three times with boiling water at 90 to 100° C., with a liquid-to-material ratio of 1 kg:10 L each time. The extract was concentrated to approximately 1 liter at 95° C., yielding a crude Parmelia tinctorum polysaccharide solution. The crude Parmelia tinctorum polysaccharide solution was mixed with ethanol to a final concentration of 80% v/v, incubated at 4° C. for 24 hours, centrifuged at 4500 rpm for 15 minutes, and lyophilized (cold trap temperature −70° C., vacuum degree 10 pa) for 24 hours to obtain crude Parmelia tinctorum polysaccharides (XEP) with a yield of 8.62%;
(2) 200 mg of the XEP obtained in step (1) was dissolved in 5 mL distilled water and loaded onto a DEAE cellulose column (20 mm×20 cm) at a flow rate of 1.8 mL/min. The column was eluted successively with 200 mL distilled water and 200 mL 0.5 mol/L NaCl solution, with 7.4 mL aliquots of eluate collected in each test tube for sugar content determination by phenol-sulfuric acid method. The main fraction was dialyzed with distilled water for 24 hours (molecular weight cut-off 3500 Da, static dialysis, changing fresh distilled water every 3 hours), followed by flowing water dialysis (molecular weight cut-off 3500 Da, filling the container with tap water, placing it in a dialysis bag, and slowly adding tap water to the container until excess tap water overflows) for 24 hours. The dialyzed solution was then lyophilized (cold trap temperature −70° C., vacuum degree 10 pa) for 24 hours to collect the polysaccharides eluted with 0.5 mol/L NaCl solution, named XEP-1, with a yield of 2.48%;
(3) XEP-1 obtained in step (2) was dissolved in deionized water to a concentration of 10 mg/mL, and ethanol was slowly added to the solution until the final ethanol concentration reached 50% (v/v). The solution was stored at 4° C. for 48 hours, centrifuged at 12000 rpm for 15 minutes, and the precipitate was collected, referred to as Parmelia tinctorum polysaccharide XEP-50, with a yield of 0.57%;

XEP-1 obtained in step (2) was dissolved in deionized water to a concentration of 10 mg/mL, and ethanol was slowly added to the solution until the final ethanol concentration reached 70% (v/v). The solution was stored at 4° C. for 48 hours, centrifuged at 12000 rpm for 15 minutes, and the precipitate was collected, referred to as Parmelia tinctorum polysaccharide XEP-70, with a yield of 0.83%;

XEP-1 obtained in step (2) was dissolved in deionized water to a concentration of 10 mg/mL, and ethanol was slowly added to the solution until the final ethanol concentration reached 90% (v/v). The solution was stored at 4° C. for 48 hours, centrifuged at 12000 rpm for 15 minutes, and the precipitate collected, referred to as Parmelia tinctorum polysaccharide XEP-90, with a yield of 0.37%.

### Test Example 1

Structural Characteristics of Parmelia tinctorum Polysaccharides

The Parmelia tinctorum polysaccharides XEP-50, XEP-70, and XEP-90 obtained in Example 1 were used as test samples for the following tests.

### 1. Uniformity and Molecular Weight Determination

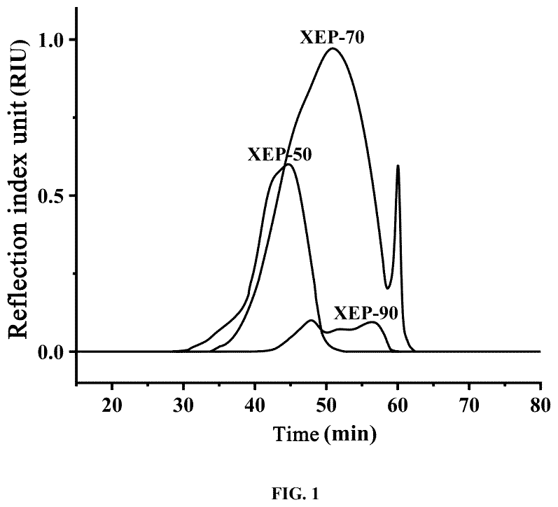

Size Exclusion Chromatography combined with Multi-Angle Laser Light Scattering and Refractive Index (SEC-MALLS-RI) was used to measure the molecular weight: Shodex OH-pak SB-805, 804, and 803 columns (inner diameter 8 mm×300 mm; Showa Denko K.K., Tokyo, Japan) were used in series; the test samples (1 mg) were dissolved in 1 mL of 0.1 mol/L NaNO₃ solution, and then filtered through a 0.45 m membrane; the column temperature was 45° C., the injection volume was 100 L, the mobile phase was 0.02% NaN₃ and 0.1 mol/L NaNO₃, and the flow rate was 0.4 mL/min; and data processing was performed using ASTRA 6.1 (Wyatt Technology, USA). The results showed that XEP-50 had only one elution peak, with a molar mass of 322.25 kDa; XEP-70 had two elution peaks, with a molar mass of 430.98 kDa; XEP-90 had two elution peaks, with a molar mass of 76.36 kDa (FIG. 1).

### 2. Chemical Composition and Monosaccharide Analysis

2.1 Using six monosaccharides as references, the total polysaccharide content was calculated using the phenol-sulfuric acid method, combined with the correction coefficients of monosaccharides (M. Dubois, K. Gilles, J. K. Hamilton, P. A. Rebers, F. Smith, A colorimetric method for the determination of sugars, Nature 168(4265) (1951) 167; F. Yue, J. Zhang, J. Xu, T. Niu, X. Lu, M. Liu, Effects of monosaccharide composition on quantitative analysis of total sugar content by phenol-sulfuric acid method, Front Nutr 9 (2022) 963318.), and the results are shown in Table 1.
2.2 Using glucuronic acid as a reference, the content of glucuronic acid was quantified using the hydroxybenzene method (N. Blumenkrantz, G. Asboe-Hansen, New method for quantitative determination of uronic acids, Anal Biochem 54(2) (1973) 484-9.), and the results are shown in Table 1.
2.3 Using bovine serum albumin as a reference material, the content of glucuronic acid was determined using the Coomassie brilliant blue method (M. M. Bradford, A rapid and sensitive method for the quantitation of microgram quantities of protein utilizing the principle of protein-dye binding, Analytical Biochemistry 72(1) (1976) 248-254.), and the results are shown in Table 1.
2.4 The test samples (3 mg) were dissolved in 1 mL of 2M trifluoroacetic acid (TFA), then sealed in ampoules at 120° C. for two hours. The remaining TFA was removed with anhydrous ethanol, and the solution was evaporated to dryness below 70° C. The polysaccharide hydrolysate was derivatized with 0.5 mL NaOH (0.3M) and 0.5 mL PMP (0.5 M) at 70° C. for 30 minutes. The mixture was neutralized with 0.5 mL 0.3M HCl and extracted three times with chloroform. The derivatives were detected using an Ultimate 3000 ultra-high performance liquid chromatography system with a Kinetex C18 column (100×4.6 mm i.d.). The process was recorded using UV absorbance at a wavelength of 245 nm, and the composition of monosaccharides was determined based on retention time and peak area, the results are shown in Table 1.

TABLE 1
Chemical composition of polysaccharides and monosaccharide
components in Parmelia tinctorum.

| | | Total | | | | | | | | |
|---|---|---|---|---|---|---|---|---|---|---|
| | Yield | sugar | Protein | Galacturonic | Monosaccharide component (%) | | | | | |
| Component | (%) | (%) | (%) | acid (%) | Man | GalUA | Glc | Gal | Xyl | Ara |
| XEP-50 | 0.57% | 89.00% | 0.91% | 1.13% | 55.00 | 2.56 | 26.48 | 13.96 | 2.00 | ND |
| XEP-70 | 0.83% | 99.05% | ND | 13.09% | 39.28 | 16.09 | 17.56 | 23.20 | 3.87 | ND |
| XEP-90 | 0.37% | 82.42% | ND | 7.22% | 32.63 | 8.64 | 15.65 | 34.67 | 1.17 | 7.24 |

Note: ND indicates not detected, and the percentage content of monosaccharides is in molar percentage.

According to Table 1, the total sugar content of XEP-50, XEP-70, and XEP-90 is 89.00%, 99.05%, and 82.42% respectively. The sugar aldehyde content of XEP-50, XEP-70, and XEP-90 is 1.13%, 13.09%, and 7.22% respectively. The protein content of XEP-50 is 0.91%, while no protein was detected in XEP-70 and XEP-90. XEP-50 mainly contains mannose (55.00%), galacturonic acid (2.56%), glucose (26.48%), galactose (13.96%), and xylose (2.00%); XEP-70 mainly contains mannose (39.28%), galacturonic acid (16.09%), glucose (17.56%), galactose (23.20%), and xylose (3.87%); XEP-90 mainly contains mannose (32.63%), galacturonic acid (8.64%), glucose (15.65%), galactose (34.67%), xylose (1.17%), and arabinose (7.24%).

### 3. FTIR Spectroscopy Analysis

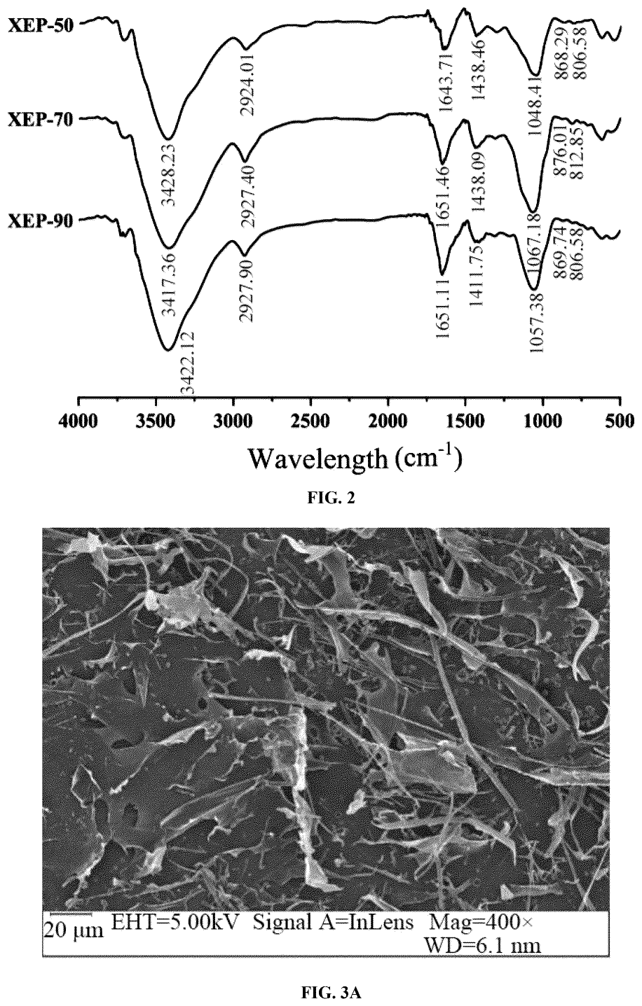

The FTIR spectra of the samples were recorded using a FTIR spectrophotometer (Nicolet-IS50, USA): the samples (2 mg) were ground with KBr powder, pelletized, and then recorded on the FTIR spectrometer (resolution, 4 cm-1), in the frequency range of 4000-400 cm-1 (mid-infrared region). The results showed that the FTIR spectra of XEP-50, XEP-70, and XEP-90 fractions displayed polysaccharide absorption peaks in the range of 4000-400 cm-1. The characteristic strong absorption band at around 3428.23 cm-1 (XEP-50), 3417.36 cm-1 (XEP-70), and 3422.12 cm-1 (XEP-90) represented the stretching vibration of O—H bonds. The stretching vibration of C—H bonds in the sugar ring was assigned to peaks at around 2924.01 cm-1 (XEP-50), 2927.40 cm-1 (XEP-70), and 2927.90 cm-1 (XEP-90). The band at 1643.71 cm-1 (XEP-50), 1651.46 cm-1 (XEP-70), and 1651.11 cm-1 (XEP-90) was due to the stretching vibration of the C═O bond in sugar aldehydes. The signals at 1438.46 cm-1 (XEP-50), 1438.09 cm-1 (XEP-70), and 1411.75 cm-1 (XEP-90) were attributed to in-plane bending vibrations of C—H bonds. The bands at 1048.41 cm-1 (XEP-50), 1067.18 cm-1 (XEP-70), and 1067.38 cm-1 (XEP-90) were attributed to the stretching vibrations of pyranose sugar rings. The bands at 868.29 cm-1 (XEP-50), 876.01 cm-1 (XEP-70), and 869.74 cm-1 (XEP-90) indicated the presence of β-glycosidic bonds in these fractions. The absorption peaks at 868.29 cm-1 (XEP-50), 876.01 cm-1 (XEP-70), and 869.74 cm-1 (XEP-90) were characteristic of mannose. It is worth mentioning that peaks at around 810 and 870 cm-1 simultaneously appeared, representing typical β-dominant configuration peaks composed of pyranose forms of glucose and mannose (FIG. 2).

### 4. Scanning Electron Microscope (SEM) Analysis

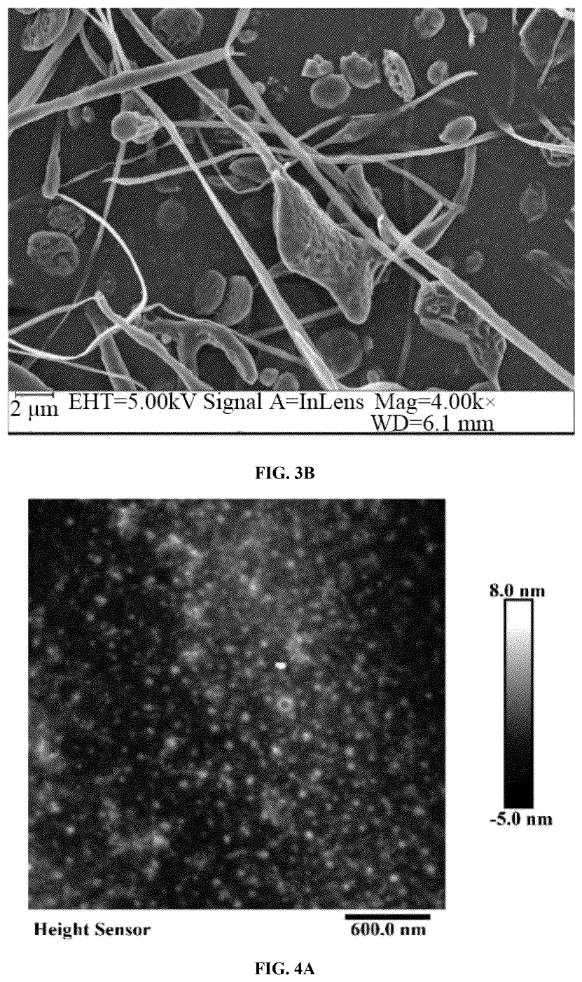

XEP-70 was placed on a conductive adhesive and sputtered with gold, and the SEM image of XEP-70 was recorded on a ZEISS GeminiSEM300 (Germany) at magnifications of 400× and 4000× under high vacuum conditions at 3.0 kV. The results showed that at 400× magnification, XEP-70 appeared as a stable irregular mesh structure. When the image was magnified to 4000×, the dense structure was composed of numerous sheet-like structures and fibrous bands (FIGS. 3A-3B).

### 5. Atomic Force Microscope (AFM) Analysis

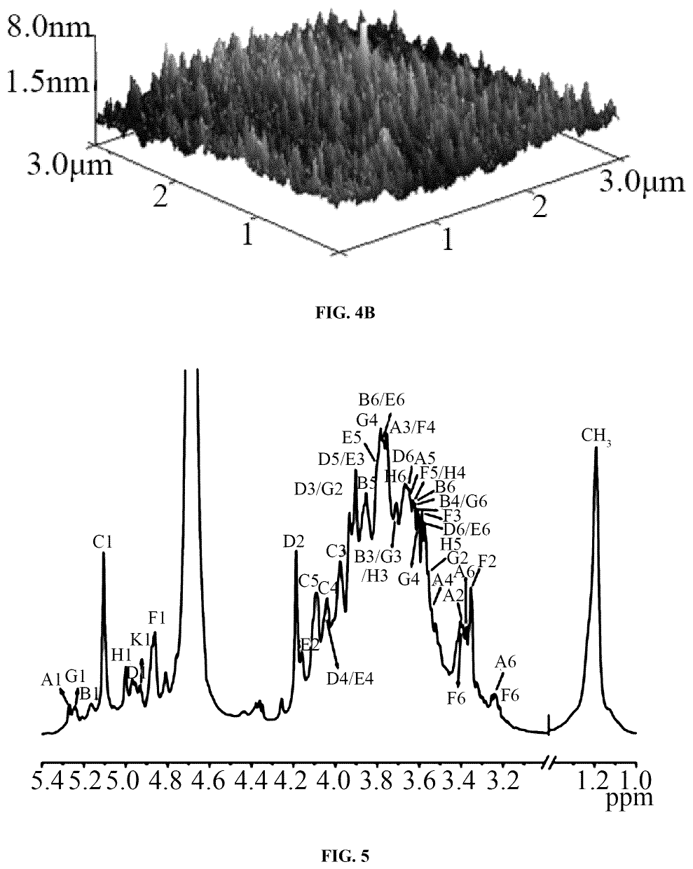

XEP-70 was dissolved in ultrapure water to a final concentration of 5 μg/mL. Then, 5 L of the polysaccharide solution was placed on the surface of a freshly cleaved mica sheet and air-dried at room temperature. The morphology of XEP-70 was detected using an atomic force microscope (Bruker Dimension Icon, Germany) with a scan size of 600 nm and a scan rate of 1.0 Hz. The atomic force microscope image of XEP-70 showed many chain-like structures. Morphological analysis indicated that XEP-70 molecules existed in the form of flexible chains, with an average thickness of 0.863 nm, between the range of single polysaccharide chains (approximately 0.1-1.0 nm) (FIGS. 4A-4B).

### 6. Nuclear Magnetic Resonance (NMR) Analysis

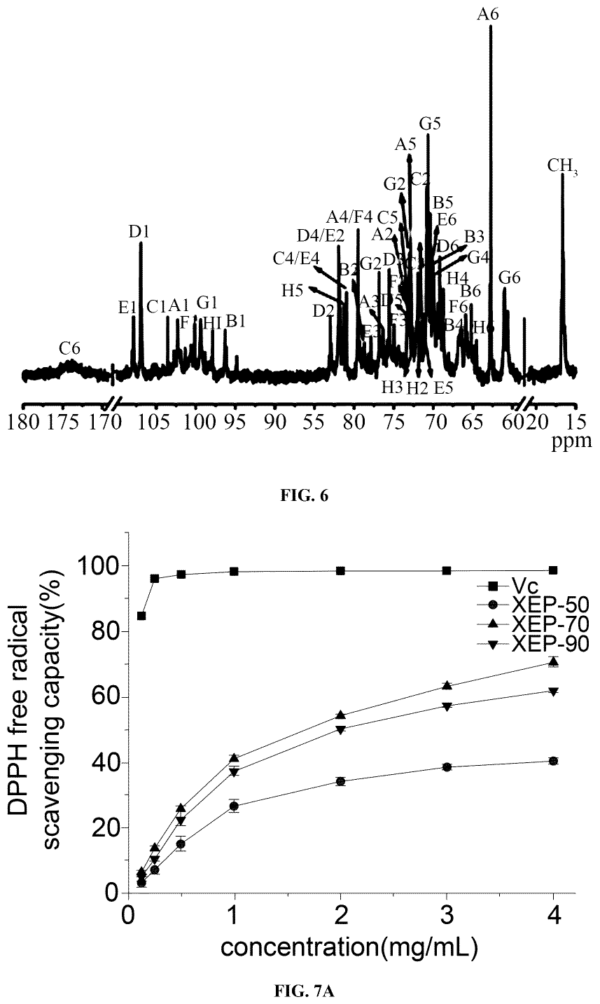

(1) XEP-70 (20 mg) was added to 0.5 mL of D₂O (99.8% D) in an NMR tube. NMR analysis, including ¹H NMR and ¹³C NMR, was monitored using a Bruker Avance 600 MHz spectrometer (Germany). The results showed that in the ¹H NMR spectrum, the region at 64.8-5.3 ppm was related to anomeric protons, with 8 signals at 5.27, 5.24, 5.16, 5.11, 5.00, 4.97, 4.93 and 4.81 ppm. Signals in the region of 63.1-4.3 ppm were attributed to H₂-H₆ (FIG. 5); the signals in the ¹³C NMR spectrum in the region of 690-110 ppm were related to isomeric carbons, with nine isomeric carbon signals observed, which could be assigned to the ¹H NMR spectrum, with signals A-H at 107.88, 106.93, 103.55, 102.30, 100.14, 99.39, 97.89 and 96.30 ppm, while the chemical shifts in the region of 660-85 ppm were related to non-isomeric carbons. The signal at 106.93 ppm was characteristic of isomeric carbons of β-galactofuranose molecules in XEP-70, as they have very low chemical shifts. The signal at 173.82 ppm was due to the presence of galacturonic acid in XEP-70 (FIG. 6).
(2) The signals of each residue of XEP-70 in the one-dimensional NMR spectrum were compared with the NMR data in the literature, and the results are shown in Table 2. Among them, the signals of B (δ5.16/96.3), G (δ5.24/99.39) and H (δ5.00/97.89) represent three different α-mannopyranose residues; the signals of A (5.27/102.30) and F (4.93/100.14) represent two different α-glucopyranose residues; the signals of D (4.97/106.93) and E (4.81/107.88) represent two different galactofuranose residues; and the signals of C (5.11/103.55) are from a galacturonic acid residue.

TABLE 2
1H and 13C NMR chemical shifts (δ): Residues A-H of XEP-70.

| | Chemical shift (ppm) | | | | | | |
|---|---|---|---|---|---|---|---|
| Retention signal | H1/C1 | H2/C2 | H3/C3 | H4/C4 | H5/C5 | H6, H′6/C6 | CH₃ |
| A | 5.27 | 3.39 | 4.09 | 3.52 | 3.65 | 3.28, 3.37 | |
| →4) -α-D-Glcp- (1→ | 102.3 | 76.85 | 75.58 | 74.96 | 72.6 | 62.73 | |
| B | 5.16 | 4.04 | 3.71 | 3.62 | 3.85 | 3.63, 3.78 | |
| →2,6)-α-D-Manp-(1→ | 96.3 | 77.97 | 69.44 | 66.58 | 70.09 | 65.9 | |
| C | 5.11 | 3.54 | 3.98 | 4.00 | 4.11 | — | 1.19 |
| →4)-α-D-GalpA-(1→ | 103.55 | 70.88 | 71.95 | 80.97 | 72.75 | 173.82 | 16.68 |
| D | 4.97 | 4.19 | 3.92 | 4.09 | 3.9 | 3.59, 3.66 | |
| →2,6)-β-D-Galf-(1→ | 106.93 | 83.03 | 75.21 | 81.94 | 73.36 | 68.9 | |
| E | 4.81 | 4.16 | 3.9 | 4.09 | 3.81 | 3.78, 3.59 | |
| →6)-β-D-Galf-(1→ | 107.88 | 81.94 | 77.89 | 80.97 | 71.17 | 69.93 | |
| F | 4.93 | 3.35 | 3.61 | 3.75 | 3.64 | 3.2, 3.4 | |
| →4,6)-α-D-Glcp-(1→ | 100.14 | 73.22 | 74.96 | 79.51 | 71.44 | 66.36 | |
| G | 5.24 | 4.04 | 3.71 | 3.6 | 3.62 | 3.86 | |
| →2)-α-D-Manp-(1→ | 99.39 | 76.85 | 72.73 | 69.19 | 70.11 | 61 | |
| H | 5.00 | 3.92 | 3.71 | 3.64 | 3.56 | 3.69 | |
| →6)-α-D-Manp-(1→ | 97.89 | 72.12 | 72.6 | 68.72 | 81.32 | 65.2 | |

According to Table 2, XEP-70 is a pectin polysaccharide composed of (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked j-D-Galf.

### Test Example 2

### Test of Free Radicals Scavenging Capacity

Free radicals are unpaired molecules in the body with strong oxidative properties. Excessive free radicals can lead to protein denaturation, cell damage, and ultimately result in diseases and aging. In this test example, the Parmelia tinctorum polysaccharides XEP-50, XEP-70, and XEP-90 obtained in Example 1, as well as vitamin C were used as test samples to describe the antioxidant activity at different concentrations, including scavenging of free radicals (ABTS, DPPH, O²⁻, and OH⁻) and reducing power. The specific steps were as follows.

### 1. DPPH Free Radical Scavenging Capacity Test

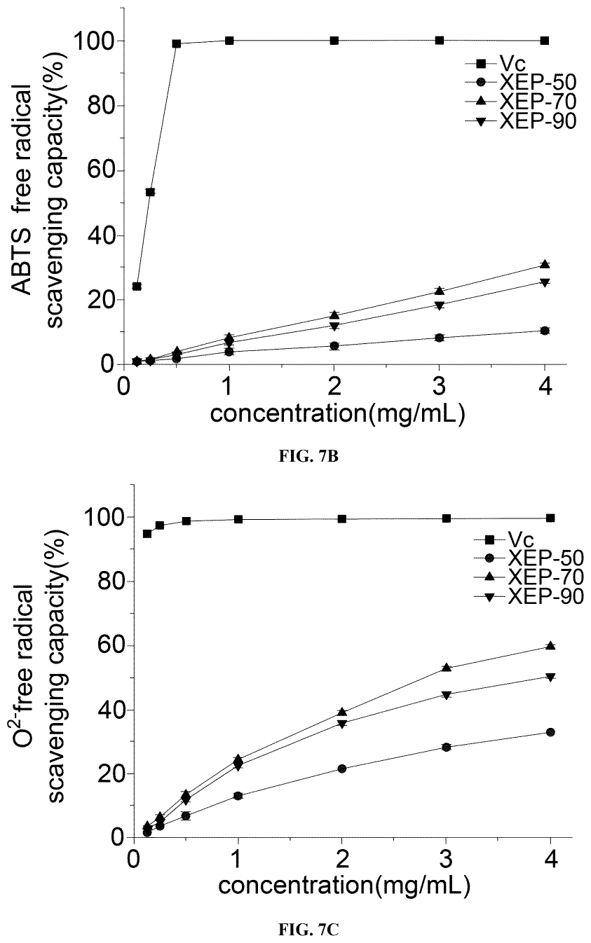

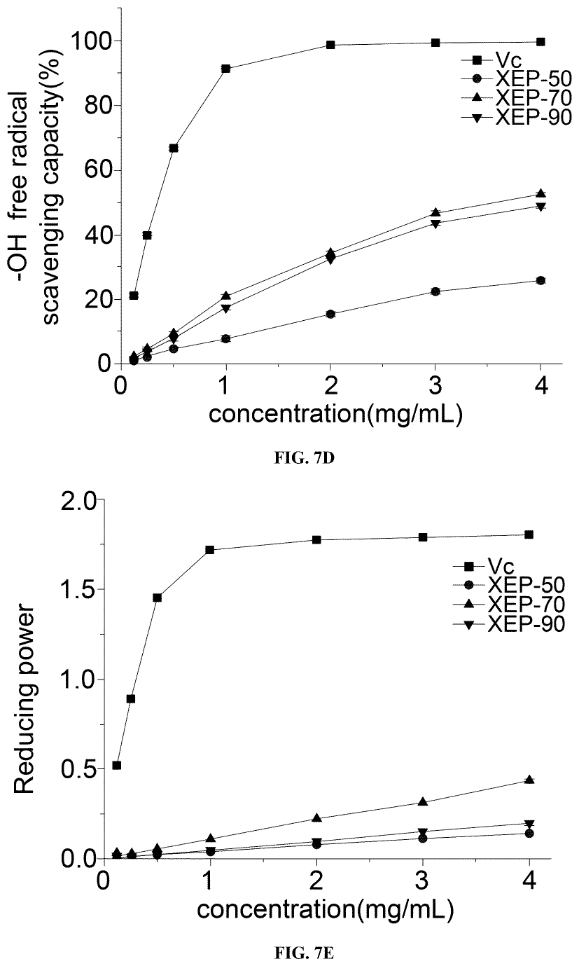

DPPH was dissolved in anhydrous ethanol to a final concentration of 0.2 mM to prepare the DPPH solution. 2 mL of the DPPH solution was mixed with 2 mL of different concentrations of the test samples. After the mixture was reacted in the dark for 35 minutes, the absorbance at 517 nm wavelength was recorded. The experiment was quadruplicated and the scavenging rate was calculated using the formula below. The results are shown in FIG. 7A.

Scavenging rate:DPPH=[1−(A₁−A₀)/A₂]*100%,

where A₀ is the absorbance of deionized water instead of the test sample solution, A₁ is the absorbance of the test sample solution, and A₂ is the absorbance of deionized water instead of DPPH and other reagents.

DPPH is a relatively stable free radical commonly used to evaluate the free radical scavenging capacity of natural compounds. As shown in FIG. 7A, within the concentration range of 0.125-4 mg/ml, the three Parmelia tinctorum polysaccharides obtained in Example 1 exhibited good DPPH free radical scavenging capacity, which increased with the concentration of polysaccharides. Among the three polysaccharides, XEP-70 showed the best free radical scavenging capacity, with a maximum scavenging rate of 70.38%±1.42%.

### 2. ABTS Free Radical Scavenging Capacity Test

To prepare the ABTS reagent, Na₂HPO₄ and NaH₂PO₄ were dissolved in deionized water (50 mM phosphate-buffered saline (PBS)). Other chemical reagents were dissolved in PBS. Using Trolox as a reference, ABTS (5 mM), HRP (1 μM), H₂O₂ (0.018%), and PBS (50 mM) solution were added to different concentrations of the test samples. After the mixture was reacted in the dark for 10 minutes, the absorbance at 730 nm wavelength was recorded. The experiment was quadruplicated and the scavenging rate was calculated using the formula below. The results are shown in FIG. 7B.

Scavenging rate:ABTS=(1−A₁/A₀)*100%,

where A₀ is the absorbance of deionized water instead of the test sample solution, and A₁ is the absorbance of the test sample solution. As shown in FIG. 7B, the increase in polysaccharide concentration is synchronized with the increase in scavenging capacity, with XEP-70 showing significantly higher scavenging efficacy compared to the other two polysaccharides.

### 3. Superoxide Radical Scavenging Capacity Test

Nitroblue tetrazolium (NBT), Nicotinamide adenine dinucleotide (reduced form, NADH), and pyridine methyl sulfate (PMS) were dissolved in deionized water, and then 1 mL of different concentrations of the test samples were added successively to the solution containing NBT (156 mol/L), with NADH (468 mol/L), and PMS (60 mol/L) each at 1 mL. After the mixture was reacted at 25° C. for 5 minutes, the absorbance at 560 nm wavelength was recorded. The experiment was quadruplicated and the scavenging rate was calculated using the formula below. The results are shown in FIG. 7C.

Scavenging rate:O²⁻=[1−(A₁−A₂)/A₀]*100%,

where A₀ is the absorbance of deionized water instead of the test sample solution, A₁ is the absorbance of the test sample solution, and A₂ is the absorbance of deionized water instead of NBT and other reagents. As shown in FIG. 7C, the three polysaccharides exhibited similar abilities to scavenge O²⁻ radicals, which increased with the concentration of polysaccharides. Among them, XEP-70 showed the best effect, reaching the maximum scavenging rate at 4 mg/ml, which was 59.34±0.60%.

### 4. Hydroxyl Radical Scavenging Capacity Test

0.5 mL of the test sample solution at three different concentrations was sequentially added with 0.5 mL of ferrous sulfate (7.5 mM), salicylic acid (5 mM), and hydrogen peroxide (10 mM). Finally, 2 mL of deionized water was added to each mixture solution. After reacting in a 37° C. water bath for 20 minutes, the absorbance was recorded at a wavelength of 510 nm. The experiment was quadruplicated. The scavenging rate was calculated according to the following formula and the results are shown in FIG. 7D.

Scavenging rate:OH⁻=[1−(A₁−A₂)/A₀]*100%,

where A₀ is the absorbance of deionized water instead of the test sample solution, A₁ is the absorbance of the test sample solution, and A₂ is the absorbance of deionized water instead of ferrous sulfate and other reagents.

According to FIG. 7D, the three polysaccharides had similar abilities to scavenge OH⁻ radicals, and the scavenging capacity increased with the concentration of polysaccharides. Among them, the effect of Parmelia tinctorum polysaccharide XEP-70 was the best, reaching the maximum scavenging rate at 4 mg/ml, which was 52.14±0.55%.

### 5. Reducing Power Test

Three different concentrations of the test samples were dissolved in 0.2M phosphate buffer (pH 6.6), and then 2.5 mL of the test sample solution at different concentrations was mixed with 2.5 mL of potassium ferricyanide (1% w/v). After reacting in a 50° C. water bath for 20 minutes, 2.5 mL of trichloroacetic acid (10% w/v) was added, the mixture solution was centrifuged (3000 rpm, 20 minutes). Then, 2.5 mL of the supernatant of each solution was added to 2.5 mL of deionized water and 250 μL of ferric chloride (0.1% w/v), and incubated for 10 minutes. The absorbance was recorded at 700 nm wavelength. The experiment was quadruplicated. The reducing power was calculated according to the following formula and the results are shown in FIG. 7E.

Reducing power=A1,

where A1 is the absorbance of the test sample solution.

According to FIG. 7E, the trend of changes in reducing power of the three polysaccharides was similar to the trend of changes in free radical scavenging capacity mentioned above, but the reducing power exhibited was not as significant as the free radical scavenging ability. Combining FIGS. 7A-7E, it can be seen that all three polysaccharides had certain free radical scavenging capacity and reducing power, among which the antioxidant activity of Parmelia tinctorum polysaccharide XEP-70 was the strongest.

### Test Example 3

### Antioxidant Activity Detection

### 1. Incubation of LX-2 Cells

LX-2 cells were grown in high glucose DMEM containing 20% (v/v) FBS, 1% (v/v) streptomycin and penicillin, at a cultivation temperature of 37° C. with 5% CO₂ in the incubator.

2. Toxicity Test of Parmelia tinctorum Polysaccharide XEP-70

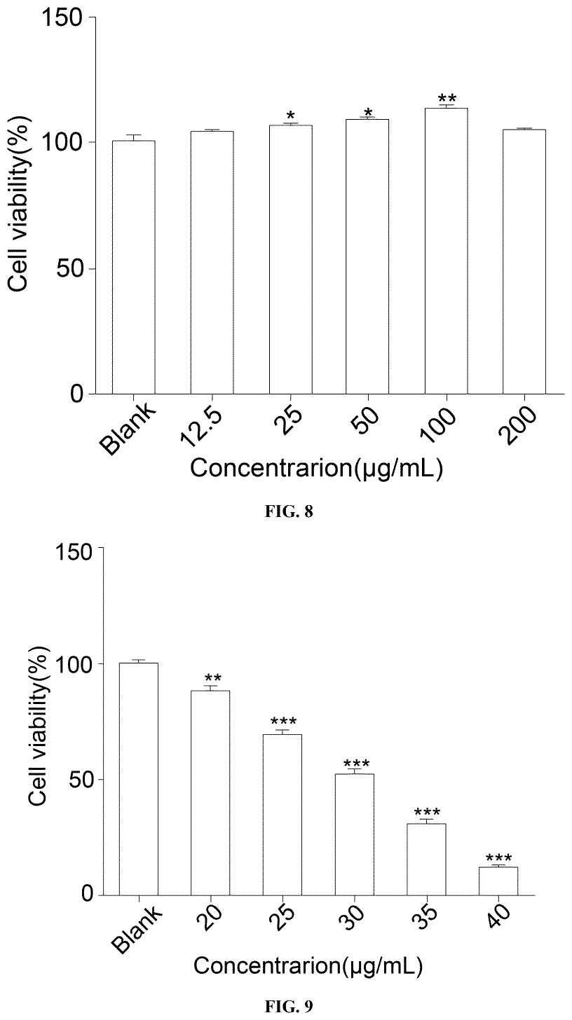

LX-2 cells were seeded in a 96-well cell plate at a density of 5000 cells/well. The Parmelia tinctorum polysaccharide XEP-70 obtained from Example 1 was added to each well to achieve final concentrations of 12.5, 25, 50, 100, or 200 μg/mL. After 24 hours of incubation, the cells were added with cell counting kit-8 (CCK-8), incubated for 1 hour, and the absorbance was measured to calculate cell viability. The results showed that Parmelia tinctorum polysaccharide XEP-70 significantly promoted cell proliferation in the range of 25-100 μg/mL (P<0.05) (FIG. 8). Therefore, subsequent experiments were conducted using 25, 50, and 100 μg/mL of Parmelia tinctorum polysaccharide XEP-70 to evaluate its antioxidant activity on LX-2 cells.

3. Screening of H₂O₂ Concentration

LX-2 cells were seeded at a density of 5000 cells/well in a 96-well cell plate. H₂O₂ was added to the wells at final concentrations of 20, 25, 30, 35, or 40 μg/mL and incubated for 24 hours. Cell counting kit-8 (CCK-8) was added to each well, followed by another 1-hour incubation to measure absorbance and calculate cell viability. The results showed that cell damage started at 20 μg/mL H₂O₂, and cell viability decreased with increasing concentrations (FIG. 9). Therefore, the IC50 concentration (30 μg/mL) was selected as the model concentration for oxidative damage in LX-2 cells.

4. Antioxidant Activity of Parmelia tinctorum Polysaccharide

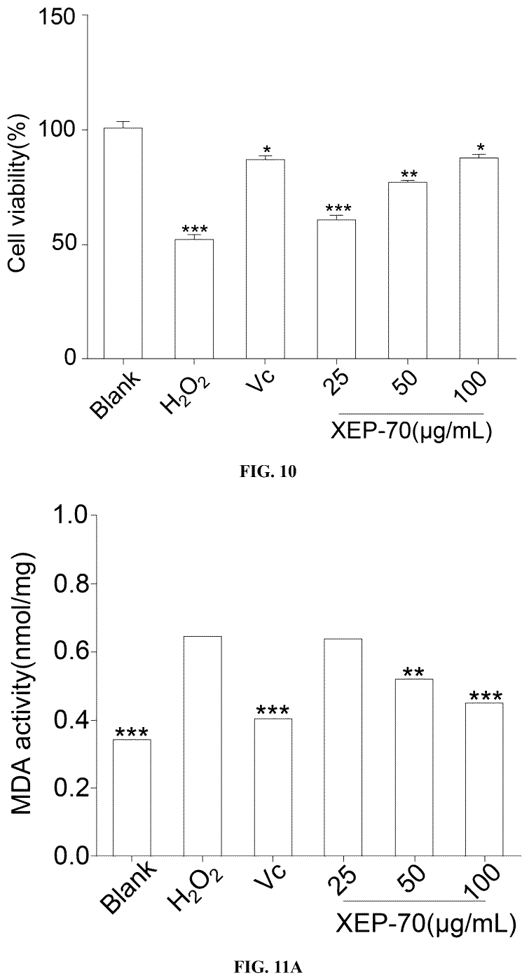

LX-2 cells were seeded at a density of 5000 cells/well in a 96-well cell plate. Parmelia tinctorum polysaccharide XEP-70 obtained from Example 1 was added to the wells at final concentrations of 25, 50, or 100 μg/mL, with Vc as a positive control (final concentration of 100 μg/mL). The cells were then incubated for 24 hours. H₂O₂ was added to the wells at a final concentration of 30 μg/mL to stimulate the cells for 24 hours, followed by a CCK8 assay to calculate cell viability. The results showed that different concentrations of Parmelia tinctorum polysaccharide XEP-70 could protect the cells and increase cell viability compared to the damage model group, with the best effect observed at 100 μg/mL, which was close to the positive control group (FIG. 10).

### 5. Antioxidant Activity and Metabolic Content Testing

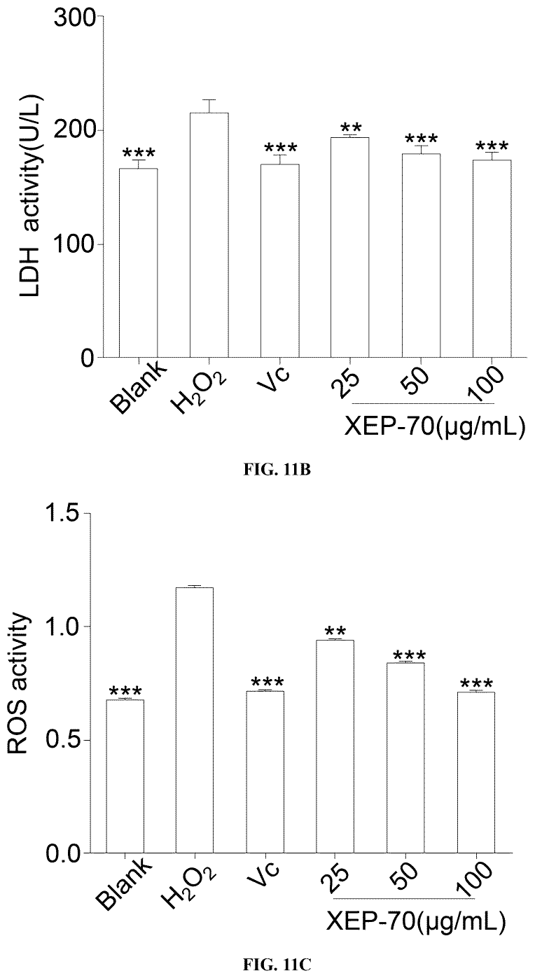

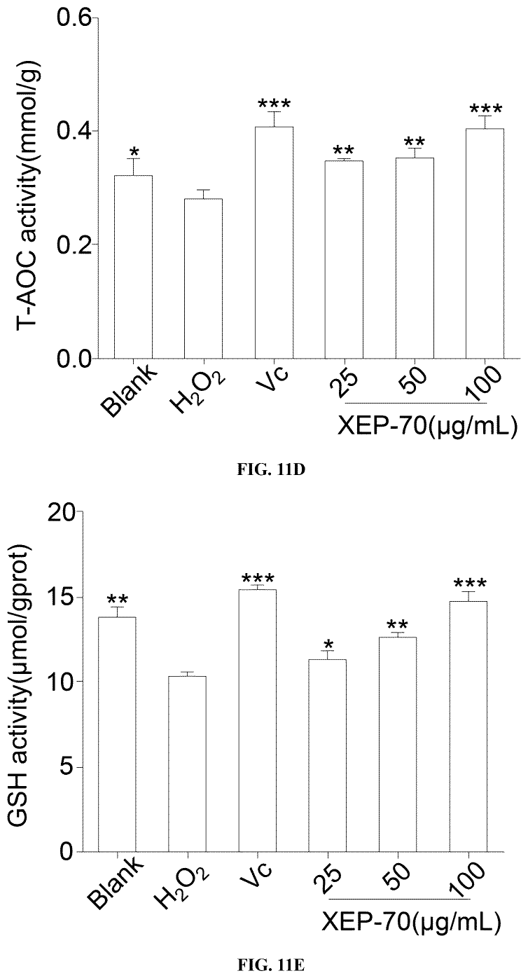

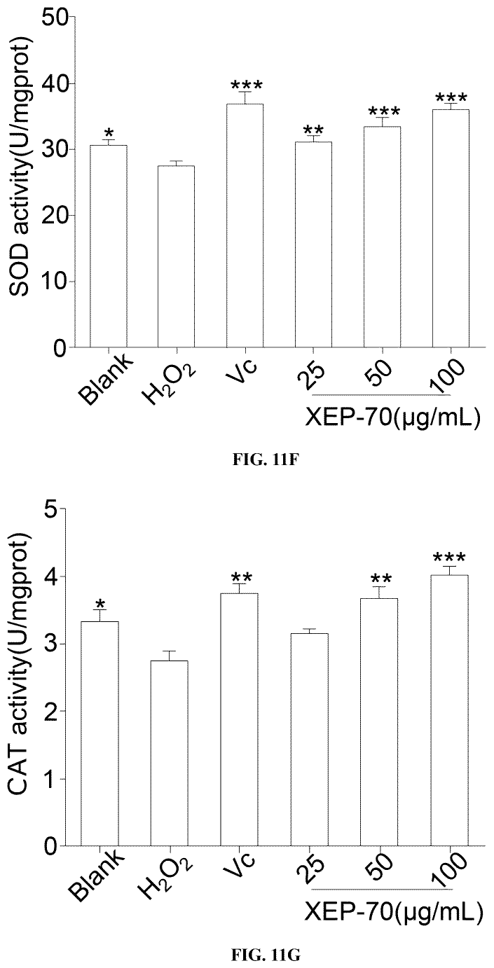

LX-2 cells were seeded at a density of 2×105 cells/well in a 6-well cell plate, with random allocation to blank group (Control), positive control group (Vc), model group (H₂O₂), and experimental group (XEP-70). Vc at 100 μg/ml was added to the positive control group, and XEP-70 at 25, 50, and 100 μg/ml was added to the experimental group, followed by a 24-hour incubation. The cells were then stimulated with H₂O₂ (30 μg/ml). After 24 hours, protein was collected, and the protein content in the cell lysate was determined using a BCA protein assay kit. The activities of T-AOC, ROS, LDH, CAT, GSH, and SOD, as well as the content of MDA, were measured in the cells. The results showed that Parmelia tinctorum polysaccharide XEP-70 could significantly reduce MDA content in the cells, decrease LDH and SOD activities, and exhibit dose-dependent effects (P<0.05). The activities of T-AOC, GSH, SOD, and CAT were significantly lower in the model group compared to the blank group, but in the experimental group pre-treated with XEP-70, the activities of these enzymes were dose-dependently reversed and significantly enhanced (P<0.05). Overall, the results indicated that Parmelia tinctorum polysaccharide XEP-70 could protect LX-2 cells from H₂O₂-induced oxidative damage by modulating the activities of antioxidant enzymes (FIGS. 11A-11G).

### 6. Western Blotting

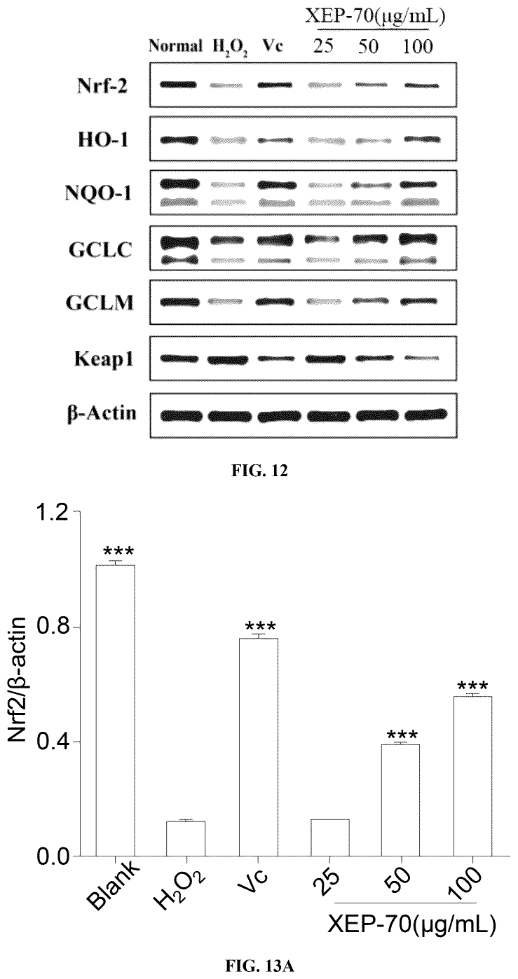

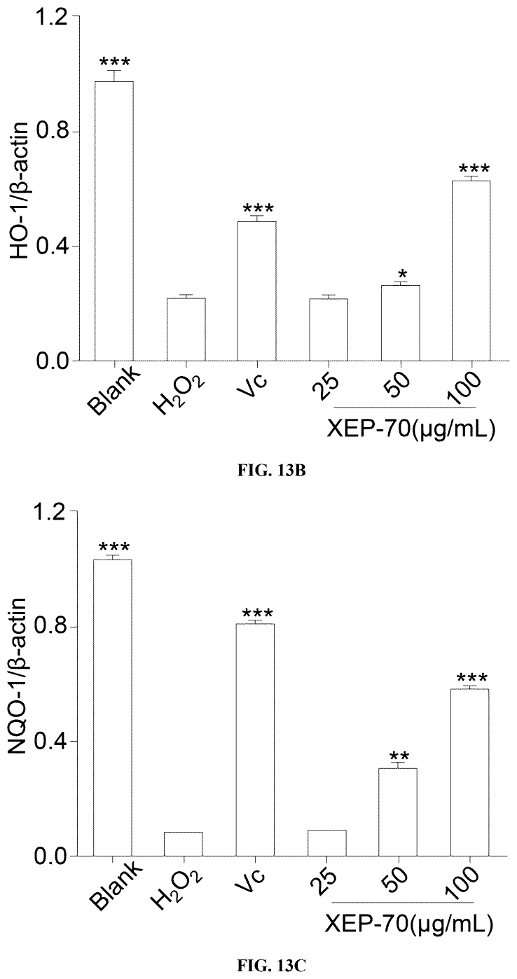

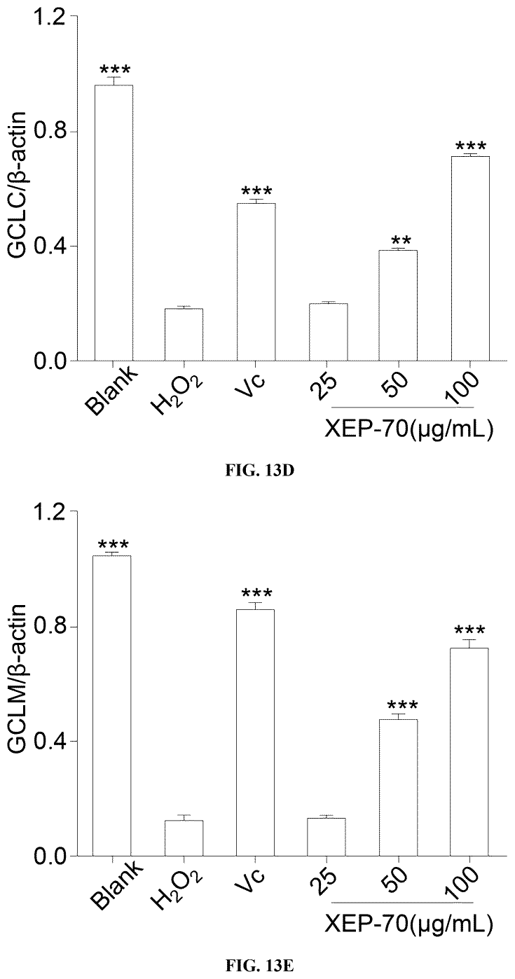

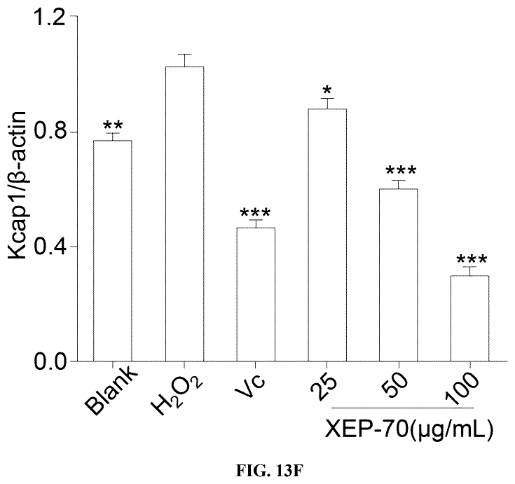

Cells were collected and processed as described in step 5. The protein lysate was separated by 10% SDS-PAGE and transferred to a PVDF membrane, followed by blocking with blocking solution for 1 hour at room temperature. The membrane was then incubated with the diluted secondary antibody for 30 minutes at room temperature. Fluorescent imaging was performed using an enzyme-linked chemiluminescence ECL assay kit, and the expression of key proteins in the Nrf2-Keap1-ARE signaling pathway was detected. The results showed that pre-treatment with 50 and 100 μg/mL concentrations of Parmelia tinctorum polysaccharide XEP-70 significantly increased the expression of NQO1, HO-1, GCLC, and GCLM (p<0.01) compared to the model group. Therefore, it can be concluded that Parmelia tinctorum polysaccharide XEP-70 activated Nrf2 in the Nrf2-Keap1-ARE signaling pathway and translocated it to the nucleus, further promoting the expression of NQO1, HO-1, GCLC, and GCLM, thereby reducing oxidative damage caused by H₂O² and exerting antioxidant effects (FIGS. 12-13). In conclusion, the Parmelia tinctorum polysaccharide XEP-70 provided in the present disclosure may enhance cellular antioxidant capacity by activating the Nrf2-Keap1-ARE signaling pathway, effectively protecting LX-2 cells from oxidative damage induced by hydrogen peroxide.

Although detailed descriptions have been provided in the above examples, they represent only a portion of the embodiments disclosed in the present disclosure, not all embodiments. Other embodiments falling within the scope of the present disclosure can be obtained without any inventive step based on the present embodiments.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. A method for preparing a Parmelia tinctorum polysaccharide XEP-70, comprising the following steps:
subjecting Parmelia tinctorum to thermal extraction, followed by solid-liquid separation and concentration to obtain a crude polysaccharide solution;
subjecting the crude polysaccharide solution to ethanol precipitation, collecting a resulting precipitate, and conducting lyophilization to obtain a crude Parmelia tinctorum polysaccharide;
performing separation and purification of the crude Parmelia tinctorum polysaccharide using a diethylaminoethyl (DEAE) anion exchange column, where the separation and purification include re-dissolving the crude Parmelia tinctorum polysaccharide in water to make an aqueous solution, loading the aqueous solution on the DEAE anion exchange column, eluting with water and 0.5 mol/L NaCl solution in sequence, collecting an eluate of 0.5 mol/L NaCl solution, subjecting the eluate to dialysis, and lyophilizing the dialyzed solution to obtain a Parmelia tinctorum polysaccharide XEP-1; and
re-dissolving the Parmelia tinctorum polysaccharide XEP-1, adding anhydrous ethanol to a final concentration of 70% v/v, allowing to stand at 0 to 4° C. for 8 to 48 h, collecting a pellet by centrifugation, subjecting the pellet to drying to obtain the Parmelia tinctorum polysaccharide XEP-70;
wherein the Parmelia tinctorum polysaccharide XEP-70 has sugar residues and glycosidic bonds comprising (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf,
wherein the Parmelia tinctorum polysaccharide XEP-70 has a weigh-average molecular weight of 430.98 kDa.

2. The method of claim 1, wherein the Parmelia tinctorum is subjected to thermal extraction with hot water three times each with a mass-volume ratio of 1 kg:10 L; and
the thermal extraction with hot water is conducted at a temperature of 90-100° C.

3. The method of claim 1, wherein the concentration performed at 95° C.; and the ethanol precipitation is conducted using ethanol at a final concentration of 80% v/v.

4. The method of claim 1, wherein the dialysis comprises distilled water dialysis with a molecular weight cut-off of 3500 Da for 24 h and flowing water dialysis with a molecular weight cut-off of 1000-10000 Da for 24 h.

5. The method of claim 4, wherein the flowing water dialysis is performed using tap water at a flow rate of 300 mL/min.

6. The method of claim 1, wherein a volume ratio of the aqueous solution, water for washing, and 0.5 mol/L NaCl solution for elution during the separation and purification is 1:40:40.

7. The method of claim 1, wherein the lyophilization is performed at a pressure of 10 to 30 MPa and a temperature of −60 to −80° C.

8. A method for preventing and/or treating oxidative damage, comprising administering to a patient in need thereof an antioxidant product comprising a Parmelia tinctorum polysaccharide XEP-70;
wherein the Parmelia tinctorum polysaccharide XEP-70 has sugar residues and (1→4)-linked α-D-GalpA, (1→4)-linked and (1→4,6)-linked α-D-Glcp; (1→2)-linked, (1→6)-linked and (1→2,6)-linked α-D-Manp; and (1→6)-linked and (1→2,6)-linked β-D-Galf,
wherein the Parmelia tinctorum polysaccharide XEP-70 has a weigh-average molecular weight of 430.98 kDa.

9. The method of claim 8, wherein the antioxidant product comprise a drug for preventing and/or treating oxidative damage.

10. The method of claim 8, wherein the drug for preventing and/or treating oxidative damage comprise a drug for preventing and/or treating oxidative damage to liver cells.

11. The method of claim 10, wherein the liver cells comprise LX-2 cells.

12. The method of claim 10, wherein the oxidative damage comprises H₂O₂-induced oxidative damage.

13. The method of claim 9, wherein the Parmelia tinctorum polysaccharide XEP-70 in the drug has an effective concentration of 25 to 100 μg/mL.

14. The method of claim 1, wherein the Parmelia tinctorum polysaccharide XEP-70 has a total sugar content of 99.05 wt. % and a glucuronic acid content of 13.09 wt. %.

15. The method of claim 1, wherein the Parmelia tinctorum polysaccharide XEP-70 consists of mannose, galacturonic acid, glucose, galactose, and xylose.

16. The method of claim 15, wherein the mannose, the galacturonic acid, the glucose, the galactose, and the xylose has molar ratio of 39.28:16.09:17.56:23.20:3.87.

17. The method of claim 8, wherein the Parmelia tinctorum polysaccharide XEP-70 has a total sugar content of 99.05 wt. %, and a glucuronic acid content of 13.09 wt. %.

18. The method of claim 8, wherein the Parmelia tinctorum polysaccharide XEP-70 consists of mannose, galacturonic acid, glucose, galactose, and xylose.

19. The method of claim 18, wherein the mannose, the galacturonic acid, the glucose, the galactose, and the xylose has a molar ratio of 39.28:16.09:17.56:23.20:3.87.
